(19) United States
(12) Patent Application Publication
(10) Pub. No.: US 2024/0150748 A1
(43) Pub. Date: May 9, 2024

(54) ACOUSTIC MICROFLUIDIC SYSTEM FOR CELL FUSION, PREPARATION METHOD THEREFOR AND USE THEREOF

(71) Applicant: SHENZHEN INSTITUTES OF ADVANCED TECHNOLOGY CHINESE ACADEMY OF SCIENCES, Shenzhen (CN)

(72) Inventors: Long MENG, Shenzhen (CN); Xiufang LIU, Shenzhen (CN); Hairong ZHENG, Shenzhen (CN); Lili NIU, Shenzhen (CN); Ning RONG, Shenzhen (CN)

(21) Appl. No.: 18/543,140

(22) Filed: Dec. 18, 2023

Related U.S. Application Data
(63) Continuation of application No. PCT/CN2021/136747, filed on Dec. 9, 2021.

(51) Int. Cl. C12N 13/00; C12M 1/42; C12M 3/06

(52) U.S. Cl. CPC C12N 13/00; C12M 23/16; C12M 35/04

(57) ABSTRACT

Provided is an acoustic microfluidic system for cell fusion, a preparation method therefor and use thereof, which relates to the technical field of cell fusion. The acoustic microfluidic system of the present invention comprises a signal generator, a power amplifier, a PDMS cavity, a micro-injection pump, a pipeline, an EP tube, a cell recovery container, and a bulk wave transducer/surface acoustic wave transducer. The side wall/bottom of the PDMS cavity is provided with identical microporous structures disposed in a staggered manner The system of the present invention has the advantages of extremely low heat production quantity, simple operation, high repeatability and strong stability, and is suitable for the fusion of homologous cells and non-homologous cells. The system is not only suitable for the fusion of two cells, but also for the fusion of a plurality of cells, and can be widely applied to various types of cells.

### CROSS-REFERENCE TO RELATED APPLICATIONS

This application is a continuation of International Patent Application No. PCT/CN2021/136747, filed on Dec. 9, 2021. The content of the aforementioned applications, including any intervening amendments thereto, are incorporated herein by reference.

### TECHNICAL FIELD

The present invention relates to the technical field of cell fusion, and, in particular to an acoustic microfluidic system for cell fusion, a preparation method therefor and use thereof.

### BACKGROUND OF THE INVENTION

Cell fusion is one of the most fundamental processes in growth, tissue repair and disease pathogenesis. Cell fusion begins with membrane fusion, with two or more independent lipid membranes being combined into one continuous bilayer by specific biological interactions to form heterogeneous cells or homogeneous cells. Because of the hybrid superiority of fused cells, the cell fusion techniques have become a powerful tool for various biological technologies, such as monoclonal antibody production, breeding improvement, stem cell production, functional cell transplantation and cancer immunotherapy. However, the existing cell fusion techniques have the problems of high cost, complex operation, high cytotoxicity, low fusion efficiency, easy inactivation of biological functions and the like. Therefore, the development of a cell fusion technique with convenient operation, high efficiency, high activity and complete biological functions has potential application values in the aspects of gene localization, tumor cell vaccines, monoclonal antibody production, animal breeding, and cancer treatment.

Currently, the most commonly used fusion techniques include polyethylene glycol fusion, viral fusion, electrofusion, magnetic fusion, and light fusion.

The polyethylene glycol fusion method is as follows: the electronegative characteristic of polyethylene glycol is utilized, such that polyethylene glycol can be combined with a positively charged cell surface group to contact adjacent cells; polyethylene glycol can change the biological membrane structure of cells, such that lipid molecules of plasma membranes at the cell contact points are dispersed and recombined, and the cells are fused due to the mutual affinity of the bilayer plasma membranes at the interface of the two cells and the surface tension of the bilayer membranes, thereby fusing the cells to form hybrid cells. Although the technique does not need special device and has low cost, the fusion rate is low, and the wide application of the technique is limited due to high cytotoxicity.

The inactivated virus fusion method is as follows: the glycoprotein and some enzymes on the surface of the virus are utilized to react with the glycoproteins on the cell membranes, such that the cells are mutually condensed, and the proteins and lipid molecules on the cell membranes are rearranged, thereby further promoting the opening of the cells and fusing the cells. Although the method is suitable for all types of animal cells, the application of the technique is limited due to the defects of difficult virus preparation, complex operation, large titer difference of inactivated viruses, poor experimental repeatability and low fusion rate.

The electrofusion method is as follows: under the induction of direct current pulse, pores on the surface of cell membranes are generated, such that paired cell membrane substances are rearranged, thereby further achieving cell fusion. The technique has the advantages of simple operation, easy and accurate adjustment of electrical parameters, no chemical toxicity, small damage to cells, high fusion rate and suitability for various cell types. However, a dedicated high-cost electrofusion device is required. Therefore, the application of the fusion technique is limited to a large extent.

The light fusion method is a cell fusion technique established based on the action of laser microbeams on cells, which utilizes the optical tweezers of the laser to capture and control the cells in a long distance and a non-contact way, and the plasma membranes of a contact area are punctured by the laser so as to achieve cell fusion. The method is a technique which is non-contact, single-cell operation, high-efficiency, suitable for various cells and also can be used in a lab-on-a-chip. However, in the process of researching cell fusion, the method has high operating requirements, has low fusion efficiency and is not beneficial to wide application.

The magnetic fusion method is as follows: the magnetic nanoparticles or the nanowires are co-incubated with the cells for a certain time of period to make the cells have magnetism, and under the action of a specific magnetic field, the magnetic force performs non-contact control and capture on the cells. The plasma membranes of the cell contact area are damaged by magnetic force, thereby achieving the cell fusion. The method is a non-contact technique which needs magnetic nanoparticles or nanowires to provide magnetism for cells and achieves paired cell fusion under the action of the magnetic force. Since the technique is complex to operate, the introduction of the magnetic nanoparticles and the nanowires increases the damage to cells, and the fusion efficiency is low, the wide application of the method is limited to a great extent.

### SUMMARY

In view of the problems in the prior art, the present invention is intended to provide an acoustic microfluidic system for cell fusion, a preparation method therefor and use thereof. The system of the present invention has the advantages of extremely low heat production quantity, simple operation, high repeatability and strong stability, and is suitable for the fusion of homologous cells and non-homologous cells. The system is not only suitable for the fusion of two cells, but also for the fusion of a plurality of cells, and can be widely applied to various types of cells.

In order to achieve the above objective, the present invention adopts the following technical solutions:

Provided is an acoustic microfluidic system for cell fusion, wherein the acoustic microfluidic system comprises a signal generator, a power amplifier, a polydimethylsiloxane (PDMS) cavity, a micro-injection pump, a pipeline, an EP tube, a cell recovery container, and a bulk wave transducer/surface acoustic wave transducer;
wherein the signal generator is used for providing sine wave signals for the bulk wave transducer; the power amplifier is used for amplifying the energy of the signals generated by the signal generator; the micro-injection pump is used for continuously injecting liquids into the PDMS cavity; the PDMS cavity comprises an array microstructure, and when a liquid is injected into the PDMS cavity, no liquid flows into the microstructure, such that microbubbles are formed; the pipeline is used for transmitting liquids and solutions with cells; the EP tube is used for recovering fused cells; the bulk wave transducer is used for generating bulk waves, wherein the bulk waves can cause the resonance of the bubbles generated by the PDMS microstructure, the bubbles vibration can cause liquid flows in the liquids, and the corresponding shearing stress and second-order radiation force are generated by the liquid flows. The cells are captured by means of the second-order radiation force, and the cells are fused by means of the shearing stress.

The side wall/bottom of the PDMS cavity is provided with identical microporous structures disposed in a staggered manner The microporous structures can capture microbubbles of an equal radius. Under the excitation of the PZT ultrasonic transducer, the array microbubbles can vibrate simultaneously, with an identical amplitude, and the experiment verifies that the microbubble vibration is a steady cavitation process, and generates identical second-order acoustic radiation forces. Under the action of second-order radiation forces, the cells in the cavity are captured onto the surfaces of the microbubbles. The shear force can be generated during the microbubble vibration process, such that cell fusion is facilitated. The resonance of microbubble arrays generates identical shearing stresses, thereby not only greatly increasing the fusion efficiency of the cells, but also processing a larger quantity of cell samples at the same time. The cells are captured onto the surfaces of the microbubbles by means of the second-order acoustic radiation force, and the distance between the cells and the flow field can be controlled by the bulk waves, so that the shearing stresses applied to the cells is accurately controlled, thereby achieving the accurate control on the capture, pairing and fusion of the cells.

For the acoustic microfluidic system for cell fusion, the PDMS cavity is embedded on a clean transparent material, preferably the transparent material comprises a glass slide, a cover slide or lithium niobate, the shape of the microporous structures of the PDMS cavity comprises a circle, an ellipse or a rectangle, and one or more of PDMS cavities are arranged in parallel. The transparent material used in the present invention not only serves as a propagation medium, but also is convenient for a microscope to observe the microbubble vibration and cell fusion processes in real time.

For the acoustic microfluidic system for cell fusion, the cell fusion comprises homologous/non-homologous cell fusion, the number of cells comprises two or more, and the cell fusion is achieved by resonance of microbubble arrays or by controlling an energy of an input signal.

Provided is a preparation method for the PDMS cavity according to any one of the embodiments described above, which comprises the following steps:
(1) pretreatment: taking a silicon substrate, removing surface impurities, and air drying the silicon substrate in a clean place;
(2) gluing and pre-baking: spin coating a negative photoresist, and horizontally placing the silicon substrate on a heating plate at 80-90° C. for baking for 0.5-2 hours; and volatilizing the solvent in the photoresist to enhance the adhesion between the photoresist and the silicon substrate;
(3) exposure and development: placing a film with a specific shape on the silicon substrate subjected to gluing and pre-baking obtained in the step (2), performing exposure and then soaking the silicon substrate in a developing solution, and after development, placing the silicon substrate on a heating plate at 130-160° C., preferably 150° C., for baking for 5-20 minutes, preferably 10 minutes;
(4) PDMS casting: uniformly mixing a glue A and a glue B of the PDMS, placing the mixture in a same culture dish with the silicon substrate subjected to exposure, development and baking obtained in the step (3), vacuumizing to remove air bubbles in the PDMS, and placing the silicon substrate in an oven at 80-90° C. for curing for 0.5-2 hours;
(5) PDMS stripping: cutting off PDMS, completely stripping off the PDMS from the silicon substrate, and punching to manufacture an inlet and an outlet to obtain the PDMS cavity; and
(6) taking a clean transparent material, performing plasma treatment on the clean transparent material and the PDMS cavity obtained in the step (5), then downwards sticking a cavity end of the PDMS cavity onto the transparent material, and baking in an oven at 70-90° C., preferably 80° C. overnight.

For the preparation method, conditions of the negative photoresist in the step (1) are as follows: a rotation rate is 2000-4000 rpm, preferably 3000 rpm, a duration is 20-40 s, preferably 30 s, and the glue comprises SU-8 (50) with a thickness of 40-60 um.

For the preparation method, conditions for the exposure in the step (2) are as follows: a dose for the exposure is 500-700 cJ/cm², preferably 600 cJ/cm², and a duration is 20-40 seconds, preferably 30 seconds; a mass ratio of the glue A and the glue B in the PDMS in the step (4) is 9-12:1, preferably 10:1.

For the preparation method, the power for the plasma treatment in the step (6) is 100-200 W, preferably 150 W, and the duration is 1-4 min, preferably 2 min.

Provided is a using method for the acoustic microfluidic system according to any one of the embodiments described above, which comprises the following steps: injecting a cell suspension into the PDMS cavity from an inlet of the PDMS cavity by using the micro-injection pump to form uniform-sized microbubbles, transmitting sound waves into the PDMS cavity by using the bulk wave transducer/surface acoustic wave transducer to generate a shearing stress and a second-order radiation force to achieve cell capture and pairing, and fusing the cells in two stages.

For the using method, an ultrasound input power for a first stage of the two-stage cell fusion is 4.7 W or less, and an ultrasound input power for a second stage is greater than the ultrasound input power for the first stage. When the input power is 4.7 W, the ultrasonic stimulation is performed for 40 ms, the cells in the PDMS cavity can be rapidly captured and paired on the surfaces of the microbubbles, and after the ultrasonic input power is increased, the cells can be fused in a short time, with the cell fusion being independent of cell types.

Provided is use of the acoustic microfluidic system for cell fusion according to any one of the embodiments described above in cell fusion, organoid fusion and related mechanisms, 3D cell culture, specific mechanism for exploring information exchange between cells, and the realization of anti-tumor effects by body-induced cell fusion.

The preset invention can not only achieve cell fusion by using the resonance of microbubble arrays, but also can achieve homologous or non-homologous cell fusion by controlling the energy of an input signal, and can also be used for controlling organisms, polystyrene microspheres and droplets, and used in gas sensors, etc. The present invention can further research various forces and specific mechanisms supplied from the cell fusion process and functional experiments of the fused cells.

The operation principle of the acoustic microfluidic system for cell fusion of the present invention is as follows:
1. Manufacturing and bonding of a PDMS cavity. The structure of the PDMS cavity is designed by adjusting the number, width, height, position and arrangement mode of pores, and a cavity mold is manufactured by using a photoetching method. Then the PDMS cavity is manufactured through the steps of glue pouring, baking, curing, punching, etc. The cavity is bonded onto a clean glass slide by using a plasma treatment method.
2. Construction of a microbubble array resonance platform: an experiment platform is built, such that the cell fusion effect can be efficiently, quickly and safely tested in the experiment platform.
3. Formation of uniform-sized microbubbles: the prepared cell suspension with a certain concentration is injected into the PDMS cavity from the inlet of the PDMS cavity at a certain flow rate by using a syringe pump, and uniform-sized microbubbles are formed in the side wall pores of the PDMS due to the interface-liquid boundary effect.
4. Cell capture and pairing: under the PZT excitation, sound waves are transmitted into the PDMS cavity by an ultrasonic coupling agent, such that the shearing stress generated in the vibration of the microbubbles enables the cells to move along the direction of microflows, the cells are captured onto the surfaces of the microbubbles by means of the generated second-order radiation force, and meanwhile, the microbubbles enable accurate pairing.
5. Cell fusion: the process is divided into two stages. Firstly, ultrasonic parameters are adjusted to ensure that cytoplasmic membrane phospholipid components of paired cells are reassembled and arranged, such that the cells are prevented from lysis; and ultrasonic parameters are continuously adjusted to enable the cells to be in a stable paired state, and then the cell fusion process is further accelerated. Since the cell fusion is achieved based on the steady cavitation of the microbubbles, the whole system is relatively stable.
6. Selection of ultrasonic experiment parameters. The experimental procedure includes the process of cell capture and pairing and the process of cell fusion (two stages). In the cell capture and pairing process, ultrasonic parameters (the number of cells is greater than or equal to 2) with highest capturing and pairing efficiency are selected; in the first stage of the cell fusion process, ultrasonic parameters which can rapidly destroy cell membranes of the paired cells are selected; and in the second stage of the cell fusion process, parameters which can enable the paired cells to be in a stable state and accelerate the rapid fusion of the paired cells are selected. The influence of various parameters on the distribution of the shearing stresses is researched, and the shearing stress threshold of cell fusion is analyzed from the mechanical angle.
7. Fused cell activity and biofunctional analysis. The fused cells are collected from the microbubble array chip system, and the activity of the fused cells cultured for a long time is evaluated by using CCK-8 kit; and meanwhile, the proliferation of the fused cells is recorded for a long time by using a living cell workstation.

Compared with the prior art, the present invention has the following beneficial effects:
(1) The present invention provides a miniature, single-cell, high-throughput, rapid and efficient cell fusion system, which has the advantages of extremely low heat production quantity, simple operation, high repeatability and strong stability and is suitable for the fusion of homologous cells and non-homologous cells. The system is not only suitable for the fusion of two cells, but also for the fusion of a plurality of cells, and can be widely applied to various types of cells. The system combines uniform-size microbubbles by means of ultrasonic waves, achieves rapid, efficient, large-scale and high-stability cell fusion based on the steady cavitation effect of the microbubbles, and shows special advantages and huge application prospects in gene localization, tumor cell vaccines, monoclonal antibody production, cancer treatment, and animal breeding. The cell type herein has universality, and the system can be used for rapidly capturing and pairing cells and has important significance for the activity of fused cells. The distance between the cells and the flow field is accurately controlled by using the bulk waves, thereby accurately controlling the cell fusion efficiency. By means of the adjusted ultrasonic energy parameters and the transient cavitation effect of the resonance microbubbles, the cell fusion effect is achieved more quickly and more efficiently.
(2) The system can well solve the problems of special requirements on apparatuses, higher preparation conditions on samples, limitation on the number of samples and the cell type, complex operation, longer time consumption, random cell pairing, low efficiency, low cell fusion efficiency, large damage to cells and the like in the prior art, and in addition, the resonance array microbubble system can manufacture microbubbles of any size through an MEMS process, and flexibly select a single ultrasonic vibration source with corresponding resonance frequency, which shows the universality of the construction of an experimental device platform. PDMS micropore cavities with different arrangement modes, different positions, different sizes and different shapes are manufactured by using a standard MEMS method, thereby achieving the cell fusion effect with low consumption and high efficiency. In addition, the system has the advantages of high design flexibility, controllable parameters, no cell difference, high repeatability, wide application in PCR amplification, gas sensors and other experiments, and high operability and cell universality in cell perforation, cell fusion, cell division, 3D cell culture and information exchange between cells.

### BRIEF DESCRIPTION OF THE DRAWINGS

FIG. 1 is a flowchart of the manufacture of a PDMS cavity;

FIG. 2 is a structural diagram of the PDMS cavity;

FIG. 3 is a schematic structural diagram of an experimental apparatus;

FIG. 4 is a diagram of microbubble capture and cell pairing; and

FIG. 5 is a diagram of the cell fusion effect of MDA-MB-231 cells generated after the treatment by an acoustic microfluidic apparatus.

### DETAILED DESCRIPTION

The present invention is further explained below by accompanying drawings and the examples.

### Example 1

### I. Manufacturing and Bonding of a PDMS Cavity

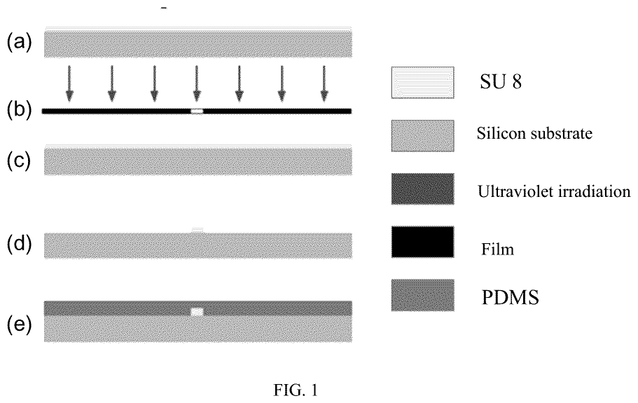

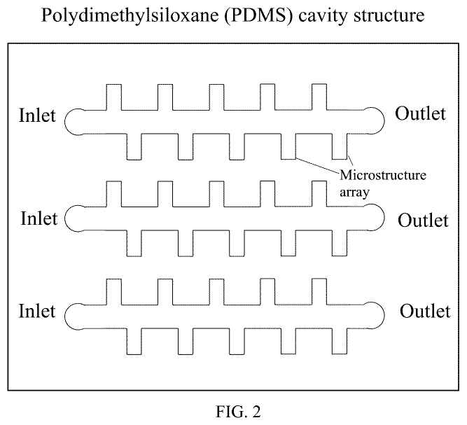

The PDMS manufacturing process is shown in FIGS. 1(a-e).
(1) Pretreatment: residual impurities, such as dust, organic adsorbates and the like, were removed from the surface of a silicon substrate by using acid washing, alcohol washing, water washing and other methods, and finally the silicon substrate was air dried in a clean place.
(2) Gluing and pre-baking: a negative photoresist SU-8 (50) was coated by spin coating with a coater at a rotation rate of 3000 rpm for 30 s, with the thickness of SU-8 (50) being about 50 μm. After the coating was finished, the silicon substrate was horizontally placed on a heating plate at 90° C. for 1 h, so as to volatilize the solvent in the photoresist, thereby enhancing the adhesion between the photoresist and the silicon substrate. The graph shown in FIG. 1(a) was obtained.
(3) Exposure and development: the patterned film as shown in FIG. 1(b) was placed on the silicon substrate as shown in FIG. 1(a), and the photoresist was exposed by using an exposure machine at a dose for the exposure of 600 cJ/cm² for 30 s, as shown in FIG. 1(c). The exposed silicon substrate was soaked in a developing solution, the photoresist of an unexposed area was dissolved, the photoresist of an exposed area was retained, and the developed silicon substrate was baked on a heating plate at 150° C. for 10 min to obtain the graph shown in FIG. 1(d).
(4) PDMS casting: a glue A and a glue B of the PDMS were uniformly mixed according to a mass ratio of 10:1, the resulting mixture was poured into a culture dish in which the silicon substrate was located, the culture dish was vacuumized to remove air bubbles in the PDMS, and finally the culture dish was placed in an oven at 80° C. for 1 h to cure the PDMS, as shown in FIG. 1(e).
(5) PDMS stripping: the patterned PDMS was cut off by using a scalpel to enable it to be completely stripped off from the silicon substrate, and finally the microcavity was punched by using a puncher to manufacture an inlet and an outlet.
(6) The PDMS cavity with a special structure (shown in FIG. 2) and a glass slide were subjected to plasma treatment, wherein the power of the plasma treatment was 150 W and the duration was 2 min, then the cavity end of the PDMS cavity was stuck downwards onto the glass slide, and then the glass slide was baked in an oven at 80° C. overnight.

### II. Construction of a Microbubble Array Resonance Platform

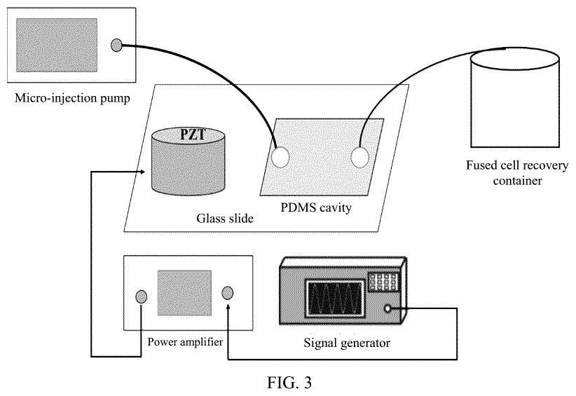

The experimental platform used in the experiment is shown in FIG. 3. The experimental platform comprises the following devices: a signal generator, a power amplifier, a PDMS cavity, a micro-injection pump, a pipeline, a cell recovery container, and a bulk wave transducer,
wherein,
1. the signal generator provided sine wave signals for the bulk wave transducer;
2. the power amplifier amplified the energy of the signals generated by the signal generator;
3. the micro-injection pump could continuously inject liquids into the PDMS cavity;
4. the PDMS cavity comprised an array microstructure, and when liquids were injected into the PDMS cavity, no liquid flowed into the microstructure, such that microbubbles were formed;
5. the pipeline was used for transmitting liquids and solutions with cells;
6. the EP tube was used for recovering the fused cells;
7. the bulk wave transducer was used for generating bulk waves, wherein the bulk waves could cause the resonance of the bubbles generated by the PDMS microstructure, the bubbles vibration could cause liquid flows in the liquids, and the corresponding shearing stress and second-order radiation force were generated by the liquid flows. The cells were captured by means of the second-order radiation force, and the cells were fused by means of the shearing stress.

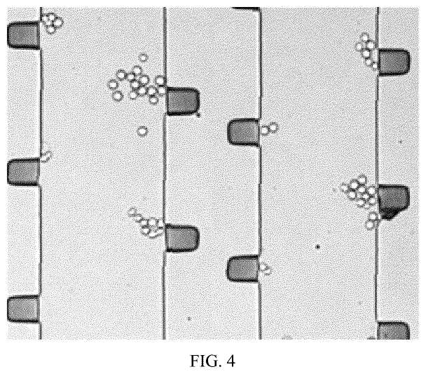

As could be seen from the result in FIG. 4, after ultrasonic treatment, two paired MDA-MB-231 cells were fused; the fluorescence field pattern showed that after the ultrasonic treatment, the cell membranes of the paired cells with fluorescent dyes were redistributed, and the fluorescence intensity of the contact area of the cell membranes was strongest, which indicated that the two MDA-MB-231 cells were fused. Therefore, it was verified that the system can achieve the cell fusion effect.

### III. Parameter Selection

The rapid, accurate and efficient pairing of cells is a key factor for achieving cell fusion. Therefore, the selection of ultrasound parameters is of crucial importance. As shown in FIG. 4, the pairing efficiency was as high as 90% at an input power of 4.7 W. Cell fusion was divided into two processes: in the first stage, cell membranes of the paired cells were destroyed rapidly and reversibly, and the fusion of cell plasma membranes was started; and in the second stage, cell fusion was accelerated and the cells were prevented from lysis. The parameters of the first stage and the parameters of the second stage were determined according to the cell perforation shearing stress threshold.

### IV. Quantitative Analysis of Activity of Fused Cells Cultured for a Long Time and Biological Functions of Fused Cells

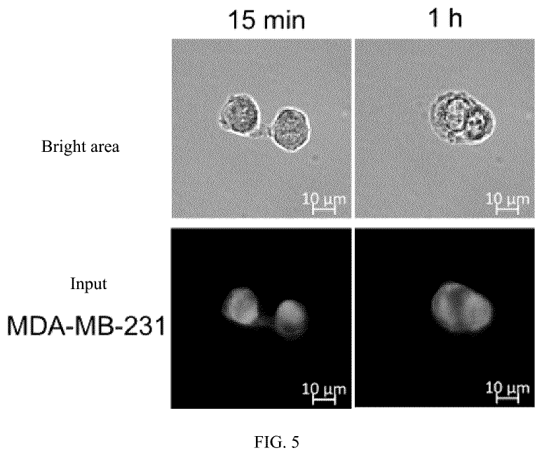

After the fused cells were collected from the PDMS cavity (see FIG. 5), the fused cells were transferred to a standard cell culture dish for culture, and the activity thereof was respectively evaluated at 24 h, 48 h, 72 h using CCK8 kit. The fused cells transferred to a 35-mm cell culture dish were placed in an inverted microscope, and the division and proliferation of the fused cells were continuously shot in a living cell workstation, such that the safety of the system was further evaluated.

What is claimed is:

1. An acoustic microfluidic system for cell fusion, wherein the acoustic microfluidic system comprises a signal generator, a power amplifier, a PDMS cavity, a micro-injection pump, a pipeline, an EP tube, a cell recovery container, and a bulk wave transducer/surface acoustic wave transducer; wherein
a side wall/bottom of the PDMS cavity is provided with identical microporous structures disposed in a staggered manner

2. The acoustic microfluidic system for cell fusion according to claim 1, wherein the PDMS cavity is embedded on a clean transparent material, preferably the transparent material comprises a glass slide, a cover slide or lithium niobate, a shape of the microporous structures of the PDMS cavity comprises a circle, an ellipse or a rectangle, and one or more of said PDMS cavities are arranged in parallel.

3. The acoustic microfluidic system for cell fusion according to claim 1, wherein the cell fusion comprises homologous/non-homologous cell fusion, a number of cells comprises two or more, and the cell fusion is achieved by resonance of microbubble arrays or by controlling an energy of an input signal.

4. A preparation method for the PDMS cavity according to claims 1, comprising the following steps:
(1) pretreatment: taking a silicon substrate, removing surface impurities, and air drying the silicon substrate in a clean place;
(2) gluing and pre-baking: spin coating a negative photoresist, and horizontally placing the silicon substrate on a heating plate at 80-90° C. for baking for 0.5-2 hours;
(3) exposure and development: placing a film with a specific shape on the silicon substrate subjected to gluing and pre-baking obtained in the step (2), performing exposure and then soaking the silicon substrate in a developing solution, and after development, placing the silicon substrate on a heating plate at 130-160° C., preferably 150° C., for baking for 5-20 minutes, preferably 10 minutes;
(4) PDMS casting: uniformly mixing a glue A and a glue B of the PDMS, placing the mixture in a same culture dish with the silicon substrate subjected to exposure, development and baking obtained in the step (3), vacuumizing to remove air bubbles in the PDMS, and placing the silicon substrate in an oven at 80-90° C. for curing for 0.5-2 hours;
(5) PDMS stripping: cutting off PDMS, completely stripping off the PDMS from the silicon substrate, and punching to manufacture an inlet and an outlet to obtain the PDMS cavity; and
(6) taking a clean transparent material, performing plasma treatment on the clean transparent material and the PDMS cavity obtained in the step (5), then downwards sticking a cavity end of the PDMS cavity onto the transparent material, and baking in an oven at 70-90° C., preferably 80° C. overnight.

5. A preparation method for the PDMS cavity according to claims 2, comprising the following steps:
(1) pretreatment: taking a silicon substrate, removing surface impurities, and air drying the silicon substrate in a clean place;
(2) gluing and pre-baking: spin coating a negative photoresist, and horizontally placing the silicon substrate on a heating plate at 80-90° C. for baking for 0.5-2 hours;
(3) exposure and development: placing a film with a specific shape on the silicon substrate subjected to gluing and pre-baking obtained in the step (2), performing exposure and then soaking the silicon substrate in a developing solution, and after development, placing the silicon substrate on a heating plate at 130-160° C., preferably 150° C., for baking for 5-20 minutes, preferably 10 minutes;
(4) PDMS casting: uniformly mixing a glue A and a glue B of the PDMS, placing the mixture in a same culture dish with the silicon substrate subjected to exposure, development and baking obtained in the step (3), vacuumizing to remove air bubbles in the PDMS, and placing the silicon substrate in an oven at 80-90° C. for curing for 0.5-2 hours;
(5) PDMS stripping: cutting off PDMS, completely stripping off the PDMS from the silicon substrate, and punching to manufacture an inlet and an outlet to obtain the PDMS cavity; and
(6) taking a clean transparent material, performing plasma treatment on the clean transparent material and the PDMS cavity obtained in the step (5), then downwards sticking a cavity end of the PDMS cavity onto the transparent material, and baking in an oven at 70-90° C., preferably 80° C. overnight.

6. The preparation method according to claim 4, wherein conditions of the negative photoresist in the step (1) are as follows: a rotation rate is 2000-4000 rpm, preferably 3000 rpm, a duration is 20-40 s, preferably 30 s, and the glue comprises SU-8 (50) with a thickness of 40-60 μm.

7. The preparation method according to claim 4, wherein conditions for the exposure in the step (2) are as follows: a dose for the exposure is 500-700 cJ/cm², preferably 600 cJ/cm², and a duration is 20-40 seconds, preferably 30 seconds; a mass ratio of the glue A and the glue B in the PDMS in the step (4) is 9-12:1, preferably 10:1.

8. The preparation method according to claim 4, wherein a power for the plasma treatment in the step (6) is 100-200 W, preferably 150 W, and a duration is 1-4 min, preferably 2 min.

9. A using method for the acoustic microfluidic system according to claims 1, comprising the following steps: injecting a cell suspension into the PDMS cavity from an inlet of the PDMS cavity by using the micro-injection pump to form uniform-sized microbubbles, transmitting sound waves into the PDMS cavity by using the bulk wave transducer/surface acoustic wave transducer to generate a shearing stress and a second-order radiation force to achieve cell capture and pairing, and fusing the cells in two stages.

10. A using method for the acoustic microfluidic system according to claims 2, comprising the following steps: injecting a cell suspension into the PDMS cavity from an inlet of the PDMS cavity by using the micro-injection pump to form uniform-sized microbubbles, transmitting sound waves into the PDMS cavity by using the bulk wave transducer/surface acoustic wave transducer to generate a shearing stress and a second-order radiation force to achieve cell capture and pairing, and fusing the cells in two stages.

11. A using method for the acoustic microfluidic system according to claims 3, comprising the following steps: injecting a cell suspension into the PDMS cavity from an inlet of the PDMS cavity by using the micro-injection pump to form uniform-sized microbubbles, transmitting sound waves into the PDMS cavity by using the bulk wave transducer/surface acoustic wave transducer to generate a shearing stress and a second-order radiation force to achieve cell capture and pairing, and fusing the cells in two stages.

12. The using method according to claim 8, wherein an ultrasound input power for a first stage of the two-stage cell fusion is 4.7 W or less, and an ultrasound input power for a second stage is greater than the ultrasound input power for the first stage.

13. Use of the acoustic microfluidic system for cell fusion according to claims 1 in cell fusion, organoid fusion, 3D cell culture or body-induced cell fusion.

14. Use of the acoustic microfluidic system for cell fusion according to claims 2 in cell fusion, organoid fusion, 3D cell culture or body-induced cell fusion.

15. Use of the acoustic microfluidic system for cell fusion according to claims 3 in cell fusion, organoid fusion, 3D cell culture or body-induced cell fusion.
